(19) United States
(12) Patent Application Publication
(10) Pub. No.: US 2020/0216221 A1
(43) Pub. Date: Jul. 9, 2020

(54) SCREEN BASKET AND COVER FOR SAID SCREEN BASKET

(71) Applicant: OLYMPUS WINTER & IBE GMBH, Hamburg (DE)

(72) Inventors: Michael WOLTER, Hamburg (DE); Ann-Kathrin Jarms, Hamburg (DE)

(73) Assignee: OLYMPUS WINTER & IBE GMBH, Hamburg (DE)

(21) Appl. No.: 16/738,182

(22) Filed: Jan. 9, 2020

(30) Foreign Application Priority Data
Jan. 9, 2019 (DE) 10 2019 100 437.3

(51) Int. Cl. B65D 21/08; A61B 50/34; A61B 50/20; B65D 43/02

(52) U.S. Cl. CPC B65D 21/083; A61B 50/34; B65D 2543/00231; B65D 43/0202; A61B 50/20

(57) ABSTRACT

A screen basket for the preparation of medical instruments. The screen basket having a substantially rectangular base area and an open upper side. The screen basket including: a first container part and a second container part, the first container part and the second container part each being open at an end face and each comprise a screen-like wall, the first container part being configured for insertion into the second container part; and at least one locking pin configured to be pushed through each of two or more push-through openings provided in one or more of the first container part and the second container part, the at least one locking pin selectively locking the first container part and the second container part together in at least two different positions with respect to each other.

### CROSS-REFERENCE TO RELATED APPLICATION

The present application is based upon and claims the benefit of priority from DE 10 2019 100 437.3 filed on Jan. 9, 2019, the entire contents of each of which are incorporated herein by reference.

### BACKGROUND

### Field

The present disclosure generally relates to a screen basket for the preparation of medical instruments and to a cover for a corresponding screen basket.

### Prior Art

Medical, and in particular surgical, instruments have to be prepared as a matter of principle after they have been used, before they are able to be used again. The preparation includes the cleaning, disinfection and possibly also sterilization of the instruments. The cleaning and disinfection can be carried out in cleaning and disinfecting devices that are realized for this purpose before the cleaned and disinfected instruments can be sterilized, where required, in a sterilizer.

Placing the medical instruments which are to be prepared by machine first of all individually or in groups in cleaning baskets which are realized as screen baskets—also called trays—before the screen basket with the instruments contained therein is moved into a cleaning and disinfecting device is known, before it is supplied, where required, to a sterilizer once the cleaning and disinfection have been carried out.

The screen basket serves, in this case, as protection against damage and recontamination of the instruments contained therein. In this case, the screen baskets from the prior art are rigid and inflexible racks. In this case, there are racks of various forms and sizes for the various application cases, each having to be made available and consequently taking up some considerable amount of storage space.

### SUMMARY

Accordingly, a screen basket is provided for the preparation of medical instruments with a substantially rectangular base area and an open upper side, including a first and a second container part, which container parts are open in each case at an end face and comprise a screen-like wall, wherein the first container part is realized for insertion into the second container part and at least one locking pin, which can be pushed through suitable push-through openings, is provided for selectively locking the telescoped container parts in at least two different positions with respect to one another.

Also provided is a cover for a screen basket including a first and a second cover part, which cover parts are arrangeable in at least two different positions with respect to one another and at least one locking pin, which can be pushed through suitable push-through openings, is provided for selectively locking the cover parts in at least two different positions with respect to one another.

A load capacity necessary for a screen basket for the preparation of medical instruments can also be achieved by two container parts which are releasably connectable together and which, furthermore, make it possible to design the screen basket in a size-modifiable manner In this case, the two container parts can be telescoped in such a manner that their respective walls combine to form a wall of a screen basket with a rectangular base area and an open upper side. At least one locking pin, which, when inserted into push-through openings which are provided for that purpose, locks the telescoped container parts together, is provided for the selective fixing of the two container parts with respect to one another. This provides for corresponding locking pins and/or push-through openings to be provided in order to be able to secure the two container parts in at least two different positions with respect to one another, which is how, ultimately, the size adjustability of the screen basket is ensured.

The at least one locking pin, in this case, can be realized non-releasably on one container part as a shaft which protrudes out of the wall thereof with a latching element at the head end, the other container part then comprising at least two push-through openings in which the at least one locking pin can be inserted. The at least one locking pin is then introduced into the push-through opening when the other container part is inserted into the one container part and then locks the two container parts with respect to one another. The number of push-through openings, in this case, is fundamentally greater than the number of locking pins, such as, at least twice as large. The locking pin can be designed in one piece with the wall or can be connected non-releasably (for example, as a result of welding or bonding) to the wall.

Alternatively, the at least one locking pin can comprise a shaft with a holding element at the foot end and a latching element at the head end so that it can be pushed through aligned push-through openings in both container parts. In this case, the at least one locking pin is therefore a separate component which can be guided into suitable push-through openings in the container parts in order to lock said container parts with respect to one another. Here too, a suitable number of push-through openings is provided in at least one container part, which number, as a rule, is greater, frequently at least twice as large as the number of locking pins.

In the event of one or multiple separate locking pins, a guide, which prevents a relative movement of the telescoped container parts perpendicularly to the open upper side, can be provided between the two container parts. In this case, it is sufficient when the at least one locking pin is able to prevent a movement along the guide. The at least one locking pin does not have to withstand any forces in the direction perpendicularly to the open upper side as said forces are absorbed by the guide.

The at least one container part can comprise at least one positioning element which allows the two container parts only to be locked in such positions in which the perforation of the wall of the two container parts is at least to the greatest possible extent free of dead space in the overlapping region. If the two container parts are connected together by the at least one locking pin, there may be a region in which the wall of the two container parts overlaps. In dependence on the design of the wall and in particular of the screen-like perforation thereof, there is the risk of the individual openings of the perforation of the two container parts not being aligned together and thus forming regions in which cleaning agent or contamination is able to collect. In so far as the formation of such dead spaces is not already effectively prevented as a result of the arrangement of the push-through openings in the two container parts, additional positioning elements can be provided on at least one of the container parts, which ensure arrangements of the two container parts with respect to one another in which no dead spaces or only very few dead spaces are formed.

The latching element of the at least one locking pin can be releasable in a toolfree manner. In other words, the two container parts should, therefore, be able to be released from one another if necessary (for example in order to be able to be joined together again for forming a different size of screen basket), without a tool being necessary to do this. The freedom from tooling simplifies the use of the screen basket in everyday medical practice. A possibility for ensuring the freedom from tooling consists in designing the latching elements in such a manner that the latching elements are released without damage in the case of a breakaway force which is greater than the forces to be expected on the latching elements when the screen basket is utilized as intended.

The push-through openings can be separate from a screen-like perforation of the wall or can be a component part of the perforation. In dependence on the design of the wall, the push-through holes can be identical to the screen openings such that separate push-through holes do not need to be provided. In this case, there is regularly such a multitude of openings present which can be utilized as push-through openings that particularly high size variability of the screen basket is achieved. However, it is also possible for the push-through openings to be formed separately from the actual screen-like perforation, for example in order to be adapted better to the design and dimensioning of the locking pins.

At least two, such as at least four locking pins can be provided. A corresponding number of locking pins has proved, on the one hand, to be sufficient for ensuring the stability of the screen basket, on the other hand to be simple to handle.

The container parts can be produced at least in part from perforated plate, wire mesh or welded wire netting. Corresponding designs can ensure the desired screen function.

The container parts and/or the at least one locking pin can be produced in a preferred manner from stainless steel.

It is also possible to arrange holders on the inside of the screen basket or of the two container parts for instruments to be prepared. The holders can be fastened releasably to the wall of the container parts in order to make it possible for the screen basket to be equipped with holding elements in a flexible manner

The cover can be designed analogously to the described screen basket, i.e. it includes two cover parts which can be locked in at least two different positions with respect to one another by means of a locking pin in order, in this way, for example, to provide a correspondingly adaptable cover for a size-adjustable screen basket. On account of the obvious analogy to the screen basket, reference is made to the preceding statements for a more detailed explanation of the cover.

### BRIEF DESCRIPTION OF THE DRAWINGS

The invention is now described in more detail as an example by way of a preferred embodiment with reference to the accompanying drawings, in which:

FIGS. 1a-1d illustrate a first exemplary embodiment of a screen basket;

FIGS. 2a-2g illustrate a second exemplary embodiment of a screen basket;

FIGS. 3a-3e illustrate a first exemplary embodiment of a cover for a screen basket similar to the embodiment of screen basket illustrated in FIGS. 2a-2g; and

FIGS. 4a-4c illustrate a second exemplary embodiment of a cover for a screen basket similar to the embodiment of screen basket illustrated in FIGS. 1a-1d.

### DETAILED DESCRIPTION

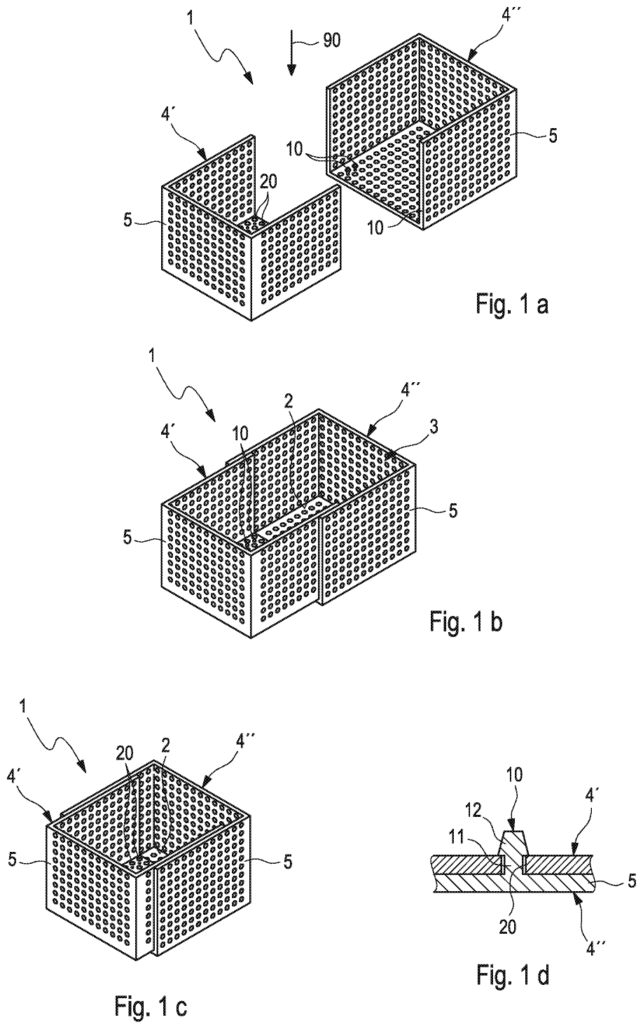

FIGS. 1a-1d show a first exemplary embodiment of a screen basket 1 for the preparation of medical instruments. In this case, FIG. 1a shows the two container parts 4′, 4″ of the screen basket 1, separated from one another, whilst in FIGS. 1b and 1c the two container parts 4′, 4″ are telescoped and locked in different positions with respect to one another. FIG. 1d shows a detail of the locking of the two container parts 4′, 4″.

The screen basket 1 for the preparation of medical instruments, with the two container parts 4′, 4″ assembled in the proper manner, comprises a rectangular base area 2 and an open upper side 3. The two container parts 4′, 4″ (the first container part 4′ and the second container part 4″) comprise, in this case, a screen-like wall 5 produced from stainless steel perforated plate and are open at one side in each case. The first container part 4′ is configured for insertion into the second container part 4″, which is why the outside dimensions thereof are adapted to the inside dimensions of the second container part 4″.

A total of four locking pins 10 (not all of which can be seen) which are configured in one piece and are consequently non-releasable from the second container part 4″, are provided on the bottom of the second container part 4″. One of the locking pins 10 is shown in detail in the sectional view according to FIG. 1d. The locking pin 10 comprises a shaft 11 which protrudes from the wall 5 of the second container part 4″, namely from the bottom thereof, and a latching element 12 is realized on the head end thereof.

Push-through openings 20, through which the locking pins 10 of the second container part 4″ can be guided and latched in, are provided on the first container part 4′. In this case, the push-through openings 20 are provided separately from the perforated plate openings and are present in sufficient numbers and arrangement in order to be able to lock the two container parts 4′, 4″ at least in the relative positions shown in FIGS. 1b and 1c. For assembling the two container parts 4′, 4″, the second container part 4″ is inserted in the direction indicated by the arrow 90 in such a manner into the first container part 4′ that the locking pins 10 are introduced into the desired push-through openings 20 and are latched therein. The latching elements 12 are configured such that they can be released in a tool-free manner as a result of applying a predefined breakaway force in the opposite direction to the direction 90.

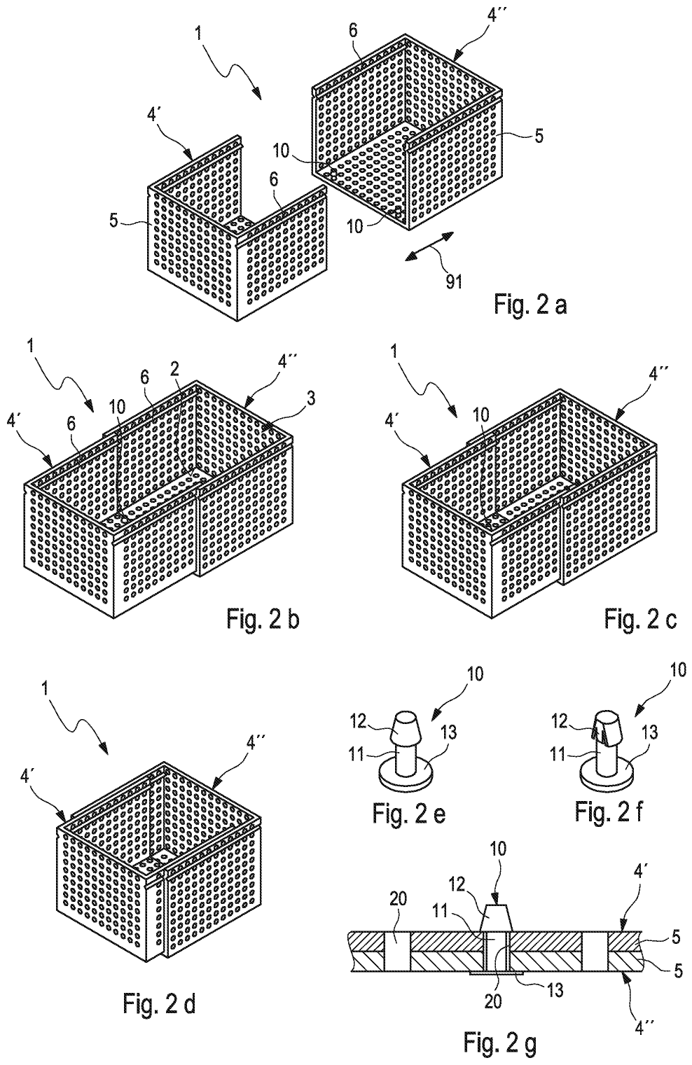

FIGS. 2a-2g show a second exemplary embodiment of a screen basket 1 for the preparation of medical instruments. FIG. 2a shows the two container parts 4′, 4″ of the screen basket 1 in a separated state, whilst FIGS. 2b, 2c and 2d show the screen basket 1 in various size configurations. FIGS. 2e and 2f show two examples of possible locking pins 10 according to the second exemplary embodiment, the use of which is outlined in FIG. 2g.

As already in the case of the exemplary embodiment according to FIG. 1, the screen basket 1 for the preparation of medical instruments, with the two container parts 4′, 4″ assembled in the proper manner, comprises a rectangular base area 2 and an open upper side 3. The two container parts 4′, 4″ which form the screen basket 1 comprise a screen-like wall 5 produced from stainless steel perforated plate and are open at one side in each case. In addition, the two container parts 4′, 4″ also comprise a guide 6 which allows the first container part 4′ to be inserted into the second container part 4″ exclusively in the direction of the arrow 91. To this end, the outside dimensions of the first container part 4′ are configured once again to the inside dimensions of the second container part 4″.

Two locking pins 10, which are configured separately from the container parts 4′, 4″, are provided for locking the two container parts 4′, 4″ with respect to one another in one of the positions shown in FIGS. 2b, 2c and 2d. FIGS. 2e and 2f show two alternative designs of corresponding locking pins 10. Both locking pins 10 include a shaft 11 with a latching element 12 at the head end. A holding element 13, which prevents the locking pin 10 from slipping through a push-through opening 20, is provided in each case at the foot end. The locking pin 10 according to FIG. 2a is produced from elastic material so that the latching element 12 is able to be compressed manually such that it can be guided through a push-through opening 20 which is adapted to the shaft 11 for releasing the locking pin 10. The locking pin 10 according to FIG. 2f is produced from stainless steel, the latching element 11 comprising two latching lugs which are also able to be compressed by a sufficient distance without any tools, however, for releasing the locking pin 10.

FIG. 2g outlines the use of a locking pin 10 according to one of FIGS. 2e and 2f. The two container parts 4′, 4″ are telescoped in the direction 91 such that the openings of the wall 5 produced from perforated plate, which at the same time provide push-through openings 20, are in alignment with one another. The locking pin 10 is then pushed through the push-through openings 20 in both container parts 4′, 4″. For release, the latching element 12 is compressed in a tool-free manner and pulled out of the push-through openings 20.

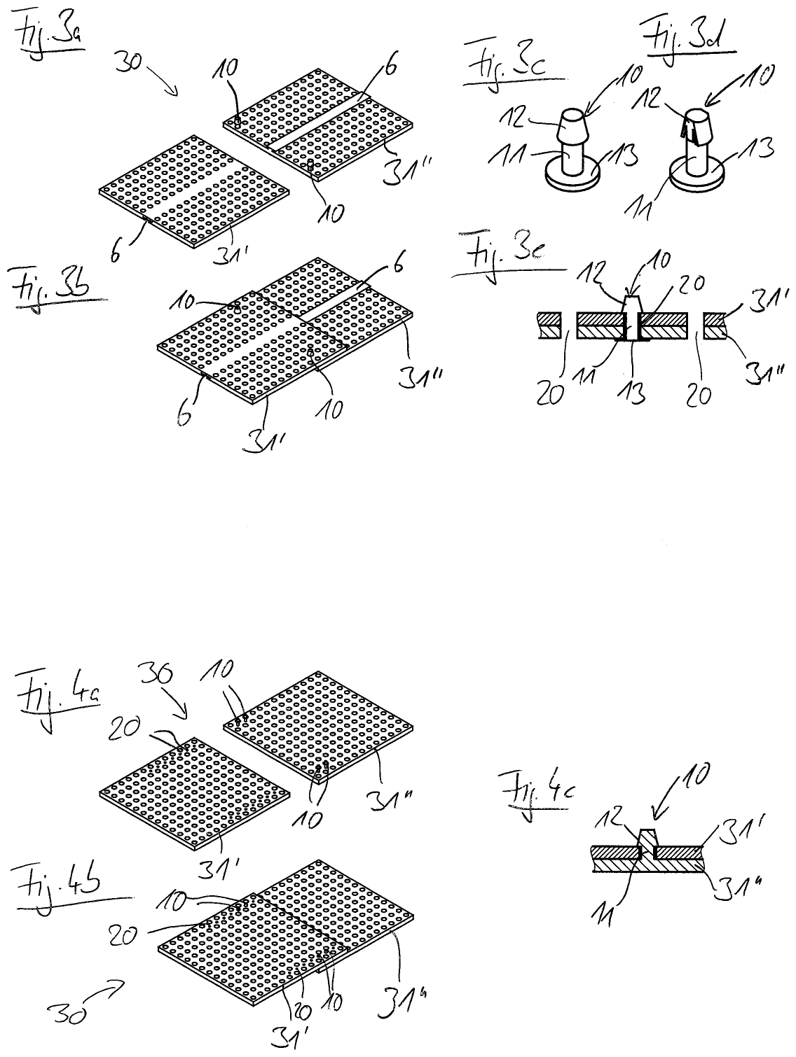

Referring now to FIGS. 3a-3e and 4a-4c, there are illustrated first and second embodiments of a cover, respectively, which can be used together with the screen baskets 1 described above. The cover, referred to in general by reference numeral 30, includes a first cover part 31′ and a second cover part 31″, which are configured similarly to the screen basket embodiments of FIGS. 1a-1d and FIGS. 2a-2g. In this regard, the cover 30 of FIGS. 3a-3e is configured similarly to the screen basket configuration illustrated in FIGS. 2a-2g and the cover 30 of FIGS. 4a-4c is configured similarly to the screen basket configuration illustrated in FIGS. 1a-1d, in which similar reference numerals are used to describe similar features. In the cover 30, the first cover part 31′ and the second cover part 31″ are arrangeable in at least two different positions with respect to one another and at least one locking pin 10, which can be pushed through suitable push-through openings, is provided for selectively locking the cover parts 31′, 31″ in at least two different positions with respect to each other. All other features described above with regard to the screen baskets 1 are also applicable to the covers 30.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A screen basket for the preparation of medical instruments, the screen basket having a substantially rectangular base area and an open upper side, the screen basket comprising:
a first container part and a second container part, the first container part and the second container part each being open at an end face and each comprise a screen-like wall, the first container part being configured for insertion into the second container part; and
at least one locking pin configured to be pushed through each of two or more push-through openings provided in one or more of the first container part and the second container part, the at least one locking pin selectively locking the first container part and the second container part together in at least two different positions with respect to each other.

2. The screen basket according to claim 1, wherein the at least one locking pin is fixed on one of the first and second container part as a shaft, the shaft protruding out of the screen-like wall with a latching element at a head end and the other of the first container part and the second container part comprises the at least two push-through openings.

3. The screen basket according to claim 1, wherein the at least one locking pin comprises a shaft with a holding element at a foot end and a latching element at a head end for pushing through aligned the at least two push-through openings in each of the first container part and the second container part.

4. The screen basket according to claim 3, further comprising a guide provided between the first container part and the second container part, the guide being configured to prevent a relative movement of the first container part and the second container part perpendicularly to the open upper side.

5. The screen basket according to claim 2, wherein the latching element of the at least one locking pin is configured to be releasable in a tool-free manner

6. The screen basket according to claim 3, wherein the latching element of the at least one locking pin is configured to be releasable in a tool-free manner

7. The screen basket according to claim 1, wherein the two or more push-through openings are formed separate from a perforation of the screen-like wall or are a component part of the perforation of the screen-like wall.

8. A screen basket according to claim 1, wherein the at least one locking pin comprises at least two locking pins.

9. A screen basket according to claim 1, wherein the first container part and the second container part are at least in part formed from one of a perforated plate, a wire mesh and a welded wire netting.

10. A screen basket according to claim 1, wherein one or more of the at least one locking pin, the first container part and the second container part are formed from stainless steel.

11. A cover for a screen basket comprising:
a first cover part and a second cover part, the first cover part and the second cover part being arrangeable in at least two different positions with respect to one another; and
at least one locking pin configured to be pushed through each of two or more push-through openings provided in one or more of the first cover part and the second cover part, the at least one locking pin selectively locking the first cover part and the second cover part in at least two different positions with respect to each other.

12. The cover according to claim 11, further comprising a guide arranged on the first cover part and the second cover part, the guide providing displacement of the first cover part relative to the second cover part.

13. The cover according to claim 11, wherein the at least one locking pin comprises a shaft with a holding element at a foot end and a latching element at a head end for pushing through an aligned one of the two or more push-through openings in both of the first cover part and the second cover part.

14. The cover according to claim 11, wherein the at least one locking pin is configured non-releasably on one of the first cover part and the second cover part as a shaft which protrudes from the screen-like wall thereof with a latching element at a head end and the other of the first cover part and the second cover part comprises the at least two push-through openings.

15. The cover according to claim 13, wherein the latching element of the at least one locking pin is configured to be releasable in a tool-free manner

16. The cover according to claim 14, wherein the latching element of the at least one locking pin is configured to be releasable in a tool-free manner

17. The cover according to claim 11, wherein the two or more push-through openings are formed separate from a screen-like perforation of the first cover part and the second cover part or are component parts of the perforation of the first cover part and the second cover part.

18. The cover according to claim 11, wherein the at least one locking pin comprises two or more locking pins.

19. The cover according to claim 11, wherein the first cover part and the second cover part are formed at least in part from a perforated plate, a wire mesh or welded wire netting.

20. The cover according to claim 11, wherein one or more of the at least one locking pin, the first container part and the second container part are formed from stainless steel.
